(19) United States
(12) Patent Application Publication
(10) Pub. No.: US 2024/0216596 A1
(43) Pub. Date: Jul. 4, 2024

(54) Medical Functional Device for Hemodialysis, Balancing Device and Method

(71) Applicants: Fresenius Medical Care Deutschland GmbH, Bad Homburg (DE); Fresenius Medical Care Holdings, Inc., Waltham, MA (US)

(72) Inventors: Peter Kotanko, Waltham, MA (US); Dejan Nikolic, Bad Soden (DE); Alexander Heide, Eppstein (DE)

(21) Appl. No.: 18/086,899

(22) Filed: Dec. 22, 2022

(51) Int. Cl. A61M 1/36; A61M 39/10

(52) U.S. Cl. CPC A61M 1/3621; A61M 39/10; A61M 2205/3331

(57) ABSTRACT

The present disclosure relates to a medical functional device or arrangement of components having a first blood circuit for extracorporally withdrawing and reintroducing blood from and to a patient (P), and a second blood circuit for extracorporally withdrawing and reintroducing blood from and to a person (D), wherein both the first blood circuit and the second blood circuit each comprise an arterial line and a venous line. In this, the medical functional device, or the arrangement, comprises at least one connecting device for establishing a fluid communication between the arterial line of the first blood circuit for the patient (P) with the venous line (73) of the second blood circuit for the person (D) and for establishing a fluid communication between the arterial line of the second blood circuit for the person (D) with the venous line of the first blood circuit for the patient (P).

### TECHNICAL FIELD

The present disclosure relates to a medical functional device, a balancing device, a blood treatment apparatus, a method for preparing components of a medical functional device for treating a patient, a method for treating a patient by using the medical functional device according to the present disclosure as well to the use of a liquid balancing device.

### BACKGROUND

Globally, 2.2 to 7.1 million patients may have died prematurely in 2010 because they did not have access to renal replacement therapy (RRT) (Source: Liyanage et al. Worldwide access to treatment for end-stage kidney disease: a systematic review, The Lancet 2015). It is apparent that social, economic, and technical barriers hinder worldwide spread of prior art RRT technology.

Therefore, a simple solution that is available to as wide a range of patients as possible is highly desirable.

### SUMMARY

It may be an object of the present disclosure to specify a further medical functional device, a balancing device, a blood treatment apparatus, a method for preparing a medical functional device for treating a patient, and a method for treating a patient by using the medical functional device according to the present disclosure or by using other objects according to the present disclosure.

Advantages according to the present disclosure may be achieved by a medical functional device, a balancing device, which may be a liquid balancing device, by a blood treatment apparatus, by a method for preparing components of a medical functional device for treating a patient, by a method for treating a patient by using the medical functional device according to the present disclosure or by using other objects according to the disclosure, and by using a liquid balancing device as described herein.

According to the present disclosure, a medical functional device (or a set or an arrangement, also referred to herein as “functional device”{) comprising components, which medical functional device comprises at least one, or exactly one, first blood circuit for extracorporeally withdrawing blood from the vascular system of a patient and for reintroducing blood into the vascular system of the patient, and which also comprises at least one, or exactly one, second blood circuit for extracorporeally withdrawing blood from the vascular system of a renally not impaired person (also referred to herein as “healthy person,” “the other person,” or “person”) and for reintroducing said blood into the vascular system of the person. Alternatively, the blood lines of the functional device or of the arrangement consist of the first and the second blood circuit.

The functional device may be used to substitute or supplement the patient's absent or missing or impaired kidney function with that of the person. In this, the functional device may in some embodiments be provided to be used together with a blood treatment component, such as a dialyzer, or, in other embodiments, without such a blood treatment component.

Therein, the first blood circuit for the patient and the second blood circuit for the person each comprise an arterial line and a venous line.

The medical functional device at least further comprises a connecting device for establishing, directly or indirectly, a fluid communication between
a) the arterial line of the first blood circuit for the patient and the venous line of the second blood circuit for the person, and
b) the arterial line of the second blood circuit for the person and the venous line of the first blood circuit for the patient.

Alternatively to a), the arterial line of the first blood circuit may be connected to, or integral with, the venous line of the second blood circuit. Alternatively to b), the arterial line of the second blood circuit may be connected to, or integral with, the venous line of the first blood circuit.

When reference is made herein to a “patient”, this refers to somebody whose blood requires treatment. This notation does not imply any information as to the gender or other characteristics of this somebody.

When the term “renally not impaired person” (also referred to herein as “person”) is used herein, it is in some embodiments intended to mean a person who is not in need of dialysis (i.e., a person whose kidneys are capable of filtering toxins and waste (urinary substances) and/or are capable of managing the regulation of salt and water balance in the body, the long-term blood pressure adjustment, and the maintenance of mineral balance). The notation “person” does not imply any information as to the gender or other characteristics of this person.

When reference is made herein to a person, it is irrelevant in some embodiments whether other diseases of the kidney, which do not impair or do not excessively impair the excretion of toxic substances and/or the regulation of the water and electrolyte balance and/or the acid-base balance, are also present in the person in question. An isolated ability of the kidney (e.g., in hormone production) does not hinder an assessment as a renally not impaired person.

Since embodiments of the present disclosure may also be used beyond dialysis, as stated herein, for example, in severe kernicterus, urea cycle defects (especially of newborns), liver failure and more, the renally not impaired person is in some embodiments, a healthy person or simply another person. These terms may herein be interchanged in some embodiments.

During dialysis or hemofiltration, liquids are supplied to the patient and/or liquids are removed from the patient. The precise balancing of these liquid quantities supplied to the patient on the one hand and removed from the patient on the other is of particular importance for the safety and health of the patient. As a result of the balancing, a balance may be drawn up of the quantities of liquid actually supplied to and removed from the patient's vascular system via the lines of the extracorporeal blood circuit, including the patient's blood being extracorporeally guided. The balance is determined, or measured, by the balancing devices. It can be documented.

Balancing devices for balancing liquid quantities are known from the field of expertise for dialysis or hemofiltration. These balancing devices are connected to the blood treatment apparatuses, which in practice are used for blood treatment, in fluid communication with the dialysate side thereof.

However, a balancing device according to the present disclosure is arranged or prepared to be arranged on both an arterial line and a venous line of a blood circuit. Said balancing device serves to determine at least one fluid balance between the arterial line and the venous line of the blood circuit, or, as will be further explained below, between the arterial line and the venous line of different blood circuits.

For this purpose, the balancing device is or will be connected, during use, to at least two flow sensors, which in turn are arranged to determine a blood flow in the arterial line and in the venous line, respectively, or said balancing device comprises such flow sensors.

Suitable holding devices for holding the line to the sensor, or vice versa, may optionally be provided, for example, as clips, recesses, etc.

The blood treatment apparatus according to the present disclosure comprises, or is connected to, at least one balancing device according to the present disclosure.

The blood treatment apparatus according to the present disclosure comprises, or is connected to, at least one medical functional device according to the present disclosure.

The method according to the present disclosure for preparing an arrangement or a set of components for treating a patient, for example by hemodialysis, hemofiltration or exchange transfusion, comprises providing a medical functional device or arrangement according to the present disclosure. It further comprises connecting the first blood circuit to the second blood circuit via the connecting device and/or connecting the first blood circuit or the second blood circuit to the flow sensors.

The method for treating a patient according to the present disclosure uses a medical functional device or arrangement according to the present disclosure and/or a blood treatment apparatus according to the present disclosure.

The use of a liquid balancing device, preferably magneto-inductive, comprising at least one or exactly one, preferably magneto-inductive, flow sensor and at least two or exactly two flow channels for differential balancing of blood is proposed by the present disclosure. Alternatively or additionally, the use of a laser-based liquid balancing device, preferably as described herein, for the differential balancing of blood is proposed.

Embodiments according to the present disclosure may comprise some, several or all of the features mentioned supra and/or in the following in any combination unless the person skilled in the art considers the particular combination to be technically impossible.

In all of the aforementioned and following statements, the use of the expression “may be” or “may have” and so on, is to be understood synonymously with “preferably is” or “preferably has”, and so on respectively, and is intended to illustrate embodiments.

Whenever numerical words are mentioned herein, the person skilled in the art shall recognize or understand them as indications of numerical lower limits. Hence, unless this leads to a contradiction evident for the person skilled in the art, the person skilled in the art shall comprehend for example “one” (or “a/an”) as encompassing “at least one”. This understanding is also equally encompassed by the present disclosure as the interpretation that a numerical word, for example, “one” (or “a/an”) may alternatively mean “exactly one”, wherever this is evidently technically possible in the view of the person skilled in the art. Both of these understandings are encompassed by the present disclosure and apply herein to all used numerical words.

The person skilled in the art shall understand spatial information like, e.g., “top”, “bottom”, “left” or “right”, whenever they are herein mentioned, as a spatial indication with reference to the alignment in the figures appended hereto and/or during use. “Bottom” is closer to the center of the earth or to the lower edge of the figure than “top”.

Whenever herein “using” is mentioned, this may in some embodiments be “a use”, and vice versa.

Advantageous developments of the present disclosure are each subject-matter of the dependent claims and embodiments.

If method steps are mentioned herein that can be performed by one of the apparatuses or devices according to the present disclosure, such as measuring, controlling, etc., then the apparatuses or devices according to the present disclosure (e.g., the functional device according to the present disclosure or the control device or closed-loop control device mentioned herein (also referred to herein as “control device”)), are in several embodiments configured in order to execute, in any combination, one, several or all of these method steps, in particular when these are automatically executable steps, or in order to accordingly control corresponding apparatuses which preferably correspond with their names to the designation of the respective method step (for example, “determining” as a method step and “apparatus for determining” for the apparatus) and which may likewise be part of the apparatus (es) or device (s) according to the present disclosure or may be connected thereto in signal communication.

When a signal communication or communication connection between two components is mentioned herein, this may be understood to mean a connection that exists during use. It may also be understood that a preparation for such a (wired, wireless or otherwise implemented) signal communication exists, for example, by coupling both components, for example by pairing, etc.

Pairing is a process that takes place in connection with computer networks in order to establish an initial link between computer units for the purpose of communication. The best-known example of this is the establishing of a Bluetooth connection, by which various devices (e.g., smartphone, headphones) are connected to each other. Pairing is sometimes also referred to as bonding.

Whenever “suitable”, “provided”, “embodied”, “configured” and/or “programmed” is mentioned herein, the person skilled in the art may understand this as a particular design of the apparatus in question. The foregoing terms may be used interchangeably herein.

Whenever an embodiment is mentioned herein, it represents an exemplary embodiment according to the present disclosure which is not to be understood as limiting.

When it is disclosed herein that the subject-matter according to the present disclosure comprises one or several features in a certain embodiment, it is also respectively disclosed herein that the subject-matter according to the present disclosure does, in other embodiments, likewise according to the present disclosure, explicitly not comprise this or these features, for example, in the sense of a disclaimer. Therefore, for every embodiment mentioned herein it applies that the converse embodiment, e.g., formulated as negation, is also disclosed.

Although embodiments of the disclosure are described below primarily with reference to the use of a dialyzer, the disclosure is not limited thereto.

The term “volume flow” as used herein may be understood as the volume of a fluid, in particular a liquid, that has moved through a cross-section, for example of the blood circuit, within a given period of time, based on the period of time required therefor. A volume flow may be given, for example, in ml per minute or ml per hour.

The term “determine” as used herein may generally mean a measuring, a detecting, a reading of a, for example, stored and/or set value and/or a calculation, in particular from known, stored, detected and/or set values.

In certain embodiments, a “line section” of the blood circuit may be understood to mean, in particular, the first (arterial) line or the second (venous) line of the extracorporeal blood circuit.

In certain embodiments, a “volume flow of the blood circuit” may be understood to mean in particular the volume flow of the first section or the volume flow of the second section of the blood circuit, in particular of an extracorporeal blood circuit.

In some embodiments of the medical functional device or the arrangement, the connecting device comprises, or consists of, one or more connectors for establishing fluid communication—directly or indirectly—between the arterial line of the first blood circuit of the patient with the venous line of the second blood circuit of the renally not impaired person (also referred to herein as “healthy person,” “other person,” or “person”) and for establishing fluid communication between the arterial line of the second blood circuit with the venous line of the first blood circuit.

Suitable adapters may be provided for establishing an indirect fluid communication. These may be encompassed by the subject-matters of the present disclosure.

In several embodiments, the medical functional device or arrangement further comprises a blood treatment component. This may be, for example, a dialyzer, a liver replacement apheresis adsorber, or a blood oxygenator, which is connected or prepared to be connected—directly or indirectly—to both the arterial line and the venous line of the first blood circuit of the patient and the arterial line and the venous line of the second blood circuit of the person.

In some embodiments, the blood treatment component (e.g., the dialyzer) is part of the connecting device. The blood treatment component, which may be a single-use item or disposable, may comprise connection sites by which it may be connected, for example by the aforementioned connector, to the arterial and venous lines, respectively, of the two extracorporeal blood circuits.

Suitable adapters, where necessary, may be provided for this purpose and encompassed by the present disclosure.

In some embodiments, the medical functional device or arrangement further comprises a first blood pump for pumping blood along the first blood circuit, particularly along the arterial line thereof.

The first blood pump and any of the pumps and further components of the medical functional device or arrangement mentioned below may be supported by a frame, housing or similar of the medical functional device; alternatively, they may be. e.g., stand-alone components.

In some embodiments, the medical functional device or arrangement further comprises a second blood pump for pumping blood along the second blood circuit, in particular along the arterial line thereof.

In several embodiments, the medical functional device or arrangement further comprises a device for variably or adjustably modifying the flow or flow area in one of the lines, or for shifting blood volume therein (e.g., a flow-pressure control component, in particular a throttle). This device may be provided in particular on or in one of the venous lines.

In addition or as an alternative to this device for variably or adjustably modifying the flow or the flow area, the medical functional device or arrangement comprises a third blood pump, which is provided for conveying blood, along the first blood circuit, in particular along the venous line thereof. In certain embodiments, it is the third blood pump that performs or supports the function of the device for variably or adjustably modifying the flow or flow area, or for shifting the blood volume.

In some embodiments, the medical functional device or the arrangement comprises a fourth blood pump, which is provided for conveying blood, along the second blood circuit, in particular along the venous line thereof. In certain embodiments, the fourth blood pump performs or supports the function of the device for variably or adjustably modifying the flow or flow area, or for shifting the blood volume.

In several embodiments, the medical functional device or the arrangement comprises a first balancing device for balancing a volume conveyed along the first blood circuit for the patient or the conveyance rate at which the conveyance was carried out.

In some embodiments, the medical functional device or arrangement comprises a second balancing device for balancing a volume conveyed along the second blood circuit for the person or the conveyance rate at which the conveyance was carried out.

In several embodiments of the medical functional device or arrangement, the first balancing device and/or the second balancing device comprises at least one flow sensor embodied and/or arranged to measure a volume or conveyance rate of the volume conveyed along the first blood circuit or along the second blood circuit, respectively.

In several embodiments of the medical functional device or arrangement, the first balancing device and/or the second balancing device is, or are prepared to be, in signal communication with the at least one flow sensor.

In some embodiments, at least one of the flow sensors is designed as a magnetic-inductive flow sensor or as a section thereof. Such inductive flow sensors are known, for example, from the application DE 10 2020 102 485 A1 of Fresenius Medical Care Deutschland GmbH, which is incorporated by reference in its entirety herein.

In several embodiments, at least one of the flow sensors is embodied as an at least dual-channel flow sensor. Such dual-channel flow sensors are described, for example, in EP 2 547 378 B1 (FIG. 2 and FIG. 3, see there) of Fresenius Medical Care Deutschland GmbH, which is incorporated by reference in its entirety herein. Channels may be measuring chambers or measuring spaces, as used herein.

In some embodiments, at least one of the flow sensors is designed as a laser-based flow sensor, ultrasound-based flow sensor, or another sensor.

In certain embodiments, the first and/or second balancing device is configured to evaluate two channels differentially or directly differentially, with a magnetic-inductive, ultrasound, or laser-based flow sensor arranged in each of the channels. In this, the flow sensors being used may be of identical type or of different types.

By using the balancing devices disclosed herein, the extracorporeal blood volume may advantageously be kept for a measurement as low as possible.

In contrast to, for example, a balancing chamber, an increase in the extracorporeally available blood volume for the measurement may not be necessary for the sake of its use.

The flow difference (input minus output) (also referred to as flow balance) of fluids or liquids may, for example, be measured directly differentially or determined by offsetting two absolute measurements of the flow. A directly differential flow measurement is more accurately or precisely (or more cost-effectively) achievable than an indirect differential measurement done by a combination of two absolute measurements whose difference is calculated, because absolute measurements are per se very complex in terms of their measurement accuracy and also more prone to error. A directly differential measurement, on the other hand, may achieve a higher accuracy with less effort, because the occurring measurement errors (per channel) of the absolute measurement cancel each other out.

In several embodiments, flow sensors may be omitted and a correspondingly simple balancing may be achieved by a single-needle method. Such a method and the necessary medical functional device or arrangement is also encompassed by the present disclosure, although this hereby involves larger blood volumes to be held extracorporeally. The same applies if alternatively two or more syringe volumes are used, which is also possible without providing flow sensors as part of the balancing device, and is encompassed by the present disclosure.

In several embodiments, at least two of the flow sensors are arranged to lie in a common magnetic field which is generated during use. A corresponding device for generating this magnetic field and the corresponding arrangement of said flow sensors in the magnetic field or future magnetic field is also subject-matter of any embodiments. Such an arrangement of the flow sensors may advantageously increase the measurement accuracy. Also, it has been observed that the effort for calibrating may thereby be reduced.

Alternatively or additionally, at least two sections of the first blood circuit and/or two sections of the second blood circuit are arranged in order to guide blood through one and the same magnetic field for at least two of the magnetic-inductive flow sensors.

Alternatively, there is provided a corresponding reception of, or receptacle for, each of the two sections of the first blood circuit and/or the second blood circuit in a common magnetic field existing during use of the medical functional device or arrangement.

In some embodiments, the first and/or the second balancing device are connected to a control device or closed-loop control device which is configured or programmed to be in signal communication, during use, with at least two of the flow sensors and to act, as a response to the signals transmitted by the flow sensors, in a closed-loop manner, on the first blood pump, the second blood pump, and/or the device (s) for variably modifying the flow. The control device or closed-loop control device may serve as a mere control device or also as a closed-loop control device. It is also referred to herein as control device.

In several embodiments, the control device is present in or on the medical functional device, for instance together with further components or devices in a common housing.

The control device is in several embodiments configured to receive measurement signals relating to the fluid balance from flow sensors in order to regulate the at least two blood pumps and the further device (s) for variably modifying the flow, wherein regulating the blood pumps and the further device (s) for variably modifying the flow is done depending on the measurement signals received or on the balance generated therefrom.

In several embodiments, the first and/or the second balancing device consist of a combination of the control device with a number of said flow sensors.

In some embodiments, the medical functional device or arrangement is embodied, at least in section, as a cassette or is present thereon or therein.

In several embodiments, the medical functional device or arrangement is embodied as a single-use item or a disposable, at least with respect to its first blood circuit (for the patient), its second blood circuit (for the person) and the flow sensors.

In some embodiments, the balancing device further comprises a control device, which in turn is configured or programmed to act, as a response to the signals transmitted by the flow sensors, in a closed-loop manner, on at least one of the blood pumps for regulating the flow through at least one of the blood circuits. Alternatively, it is connected to such a control device.

In some embodiments, the balancing device consists of a control device and flow sensors.

In several embodiments, the method according to the present disclosure for treating a patient using a medical functional device or arrangement according to the present disclosure and/or a blood treatment apparatus according to the present disclosure comprises at least the following steps:
determining at least a first volume flow in at least the arterial line of the first blood circuit for the patient or of the second blood circuit for the person by the flow sensors respectively arranged thereon;
determining at least a second volume flow in at least the venous line of the first blood circuit for the patient or of the second blood circuit for the person by the flow sensors respectively arranged thereon;
establishing a fluid balance from at least the first volume flow and the second volume flow; and
controlling and/or controlling in a closed-loop manner at least one of:
a) a volume flow of the first blood circuit for the patient or of the second blood circuit for the person, and/or
b) a pump rate of at least one blood pump of the first blood circuit and/or the second blood circuit, and/or
c) at least one device for variably or adjustably modifying the flow based on the generated fluid balance.

It is known that the requirements for the use of the functional device according to the present disclosure represent a very high hurdle to the selection of a suitable combination of patient and person, which is similarly high as in an (organ) transplantation. Examples of these are, for example, the requirements for blood group compatibility.

The number of patients mentioned above who die before reaching adulthood because they have no access to modern, high-tech renal replacement therapy sheds a different light on the high requirements.

In several embodiments, usually when a blood purification component such as those described herein is used, the requirements for the suitable combination of patient and person (e.g., for blood compatibility) are very low. This is because in these embodiments, the patient's blood does not come into contact with the blood of the person, but only toxins and/or waste substances or water pass from the patient's bloodstream into the bloodstream of the person via the blood purification component, in particular via its membrane, but what does not pass is for example the majority of the antibodies present in the blood and in particular no cells. The toxins and/or waste substances or the overhydration can be processed by the kidneys of the person and excreted via the body of the person. These embodiments further advantageously minimize or prevent the passage of infectious material and any type of immunologically active molecules, since the pore size of the membrane is generally too small for these substances. However, substances the transfer of which from the person to the patient is or can be beneficial to the patient (e.g., bicarbonate, vitamins, amino acids and the like), can pass through.

In several embodiments, the medical functional device and/or the balancing device according to the present disclosure are embodied as a tube system, tube set, cassette, in particular a blood cassette.

In some embodiments, the balancing device according to the present disclosure is embodied as a single-use item or a disposable.

The term “single-use item or disposable” as used herein may be understood to mean that the medical functional device or components thereof are intended for single use, for example in a method for the extracorporeal treatment of a patient's blood. It can be provided and/or marketed as a disposable, a single-use item, a throw-away item, or similar.

In some embodiments, the medical functional device or the balancing device comprises, or is in data/signal communication with, at least one evaluation unit or evaluation device for determining at least one fluid balance.

In some embodiments, at least two of the flow sensors are part of a differential blood balance sensor, comprising two liquid channels each having one of the flow sensors. A blood balance sensor may comprise a housing which comprises or encloses or encases more than one flow sensor.

In some embodiments, the control device is configured to regulate the at least two blood pumps and the further actuator after receiving measurement signals relating to the fluid balance (may be a liquid balance, in particular a blood balance or blood flow balance) from the balancing device, wherein regulating the blood pumps and the actuator is done depending on the received measurement signals relating to the fluid balance.

In several embodiments, the closed-loop controlling and/or controlling of the volume flow is done at least based on and/or at least by evaluating the fluid balance.

In some embodiments, the fluid balance (abbreviated here as FB), may be used as a signal and/or to generate a signal for the control device to control and/or close-loop control at least one volume flow of the blood circuit, a pump rate of at least one pump of the blood circuit, a cross-section of at least one section of the blood circuit.

In several embodiments, the fluid balance (FB) may be established from at least a first volume flow (F1) and a second volume flow (F2).

In some embodiments, the fluid balance (FB) may be formed as follows:

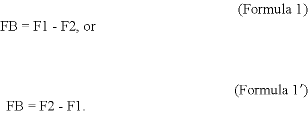

In some embodiments, the fluid balance (FB) is used as a signal and/or to generate a signal for the control device to control or to control in a closed-loop manner at least one volume flow of the blood circuit. In some embodiments according to the present disclosure, in particular the first or the second volume flow may be increased or decreased for this purpose.

In certain embodiments, the blood treatment apparatus according to the present disclosure is configured or designed as a hemodialysis apparatus, a hemofiltration apparatus, a hemodiafiltration apparatus, generally a dialysis apparatus, for example an apparatus for the acute dialysis, for the chronic dialysis or other forms of blood purification. The blood treatment apparatus may be an apparatus for performing liver replacement procedures, an apparatus for performing immunoadsorption, or similar.

In several embodiments, the medical functional device, the balancing device, and/or the blood treatment apparatus does not comprise a scale and/or a balance chamber, particularly no non-ideal, rigid, balance chamber.

In certain embodiments, the method does not comprise gravimetric monitoring, and/or the ultrafiltration rate is not directly measured with a sensor, in particular not with a magnetic-inductive flow sensor.

In several embodiments, the functional device does not comprise a blood container for the extracorporeal temporary storage, preferably not a blood container comprising a cylindrical housing.

In some embodiments, further encompassed by the present disclosure is effectively balancing to 0 by arranging two blood pumps (e.g., the first and the third) in one of the blood circuits—for example, one upstream and one downstream of, for instance, the blood treatment component (e.g., the dialyzer)—and setting both to the same conveyance rate. Then the balance (within the combined accuracies of the pumps) must be zero. In this way, embodiments of the present disclosure would require no flow sensors and no means of adjustment or the possibility to affect the balance, since it would then be a force-guided system during use. A typical use of such an embodiment of the present disclosure in this context would be a system for neonates, for example in which system the primary concern is about the exchange transfusion in connection with a kernicterus, less about the withdrawal of fluid (as a plasma fraction), e.g., overhydration. In this—in contrast to an open balancing system using the flow sensors—there remains no possibility to adjust to changing conditions (for example blood/pressure changes, which may occur spontaneously or also gradually).

In several embodiments, the arterial line and/or the venous line of the first blood circuit have connectors of a first type for connecting them to the connection sites on the one hand, and the arterial line and/or the venous line of the second blood circuit have connectors of a second type for connecting them to the connection sites on the other hand, the first type and the second type being geometrically different from each other.

In some embodiments, the medical functional device according to the present disclosure does not comprise a dialysis liquid inlet line (i.e., no inlet line for a fresh dialysis liquid), to the blood treatment component.

In several embodiments, the medical functional device according to the present disclosure does not comprise a dialysate outlet line (i.e., no outlet line for spent dialysis liquid), away from the blood treatment component.

In some embodiments, there is no balancing taking place, particularly volumetric balancing, using balancing chambers.

In several embodiments, the medical functional device does not comprise a syringe pump-based system. By using such a system, either quasi-continuous flow could not be implemented or duplicate syringes would have to be provided, thus greatly increasing the extracorporeal volume.

Some or all embodiments may comprise one, several, or all of the advantages mentioned supra and/or below.

An advantage of the present disclosure may be to dialyze or diafilter patients even under adverse conditions in which, e.g., dialysis liquid is not available and there is also no possibility for preparing dialysis liquid on site.

If even a blood treatment component, such as a dialyzer, is not available, with the present disclosure, after clarification of compatibility and other health issues, the life of the renally-impaired patient can be temporarily saved and/or sustainably prolonged by resorting to the (renally not impaired/healthy) person.

Another advantage of embodiments of the present disclosure may be that by the balancing device, which does not require balancing chambers and which establishes a fluid balance based on volume flows determined via flow sensors, it is possible to avoid holding or keeping or temporarily storing blood outside the patient's body or the body of the person. The volume of blood withdrawn is minimized and thus a drop in blood pressure caused by blood volume withdrawal is prevented or at least counteracted. The time period of the blood being kept outside the body is minimized according to the present disclosure, whereby clotting and/or blood damage can be avoided.

Another advantage of embodiments of the present disclosure may be that due to the proposed balancing with the quality of tube and blood pumps the need for excessively expensive components may be omitted. This may help to save costs. The required accuracy may be achieved by the flow control.

All or some of the advantages achievable with the methods according to the present disclosure may, in certain embodiments, also be achieved to an undiminished extent with the devices according to the disclosure (i.e., the medical functional device according to the present disclosure, the balancing device according to the present disclosure and/or the blood treatment apparatus according to the present disclosure), and vice versa.

Advantageously, precise balancing is enabled in some embodiments.

According to the present disclosure, it is further possible to even allow the patient to benefit from a blood treatment as described herein in the event that although a blood treatment apparatus is available but dialysis liquid is not available due to particular reasons. The dialysis or filtration required to treat the effects of renal insufficiency on the patient's blood can be accomplished by embodiments of the present disclosure while incorporating the functioning renal function of the person.

### BRIEF DESCRIPTION OF THE DRAWINGS

In the following, embodiments of the present disclosure will be merely exemplarily described with reference to the accompanying figures. In them, same reference numerals denote identical or similar components. In the figures the following applies:

FIG. 1 shows in a simplified representation a medical functional device according to the present disclosure or an arrangement of components thereof in a first embodiment;

FIG. 2 shows in a simplified representation a medical functional device according to the present disclosure or an arrangement of components thereof in a further embodiment during use;

FIG. 3 shows in a simplified representation the course of a method according to the present disclosure for treating a patient using the medical functional device in an embodiment; and

FIG. 4 shows in a simplified representation the course of a method according to the present disclosure for preparing an arrangement of components for treating a patient by hemodialysis or exchange transfusion.

### DETAILED DESCRIPTION

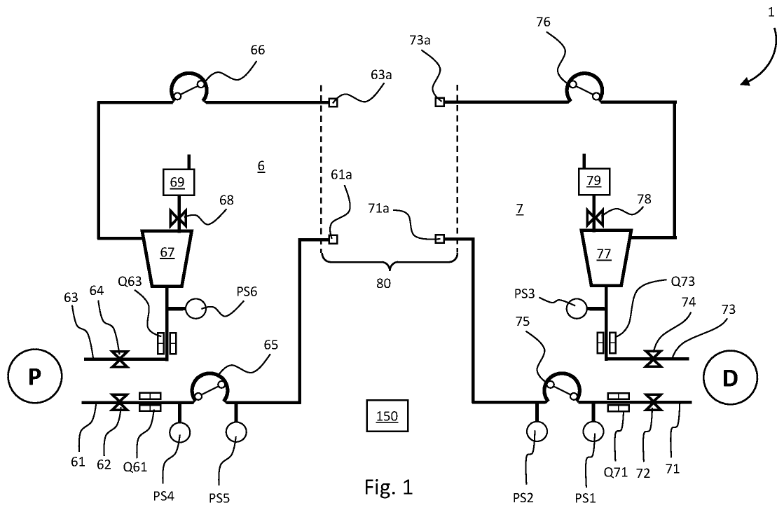

FIG. 1 shows in a simplified representation a medical functional device 1 according to the present disclosure or an arrangement of components thereof in a first embodiment.

The medical functional device 1 comprises at least, or exactly, one first extracorporeal blood tube set or extracorporeal blood circuit 6 for extracorporeally withdrawing blood from a patient P, who is only outlined in FIG. 1 but not connected, from their vascular system and for reintroducing this blood into the vascular system of the patient P still to be connected.

Further, the medical functional device 1 comprises a second extracorporeal blood tube set or extracorporeal blood circuit 7 for extracorporeally withdrawing blood from a healthy person, in particular a renally not impaired person D (also referred to herein as “person”), which is only outlined in FIG. 1 but not connected, and for reintroducing this blood into the vascular system of the person D still to be connected.

In this, both the first blood circuit 6 and the second blood circuit 7 each have an arterial line 61 or 71 (also: arterial line section) and a venous line 63 or 73 (also: venous line section).

Furthermore, the medical functional device 1, or the arrangement, which herein may also be understood as a set of components, comprises at least one connecting device 80, which may be suitable or provided for, directly or indirectly, establishing a fluid communication between the arterial line 61 of the first blood circuit 6 of the patient P with the venous line 73 of the second blood circuit 7 of the person D, and for establishing, directly or indirectly, a fluid communication between the arterial line 71 of the second blood circuit 7 of the person D with the venous line 63 of the first blood circuit 6 of the patient P.

In the example of FIG. 1, the connecting device 80 comprises or consists of connectors 61a, 63a, 71a, 73a, suitable for this purpose, which are arranged on, are part of or are associated with the lines 61, 63, 71 or 73, respectively.

Alternatively, the arterial line 61 of the first blood circuit 6 of the patient P may be connected to, or integral with, the venous line 73 of the second blood circuit 7 of the person D, and likewise the arterial line 71 of the second blood circuit 7 of the person D may be connected to the venous line 63 of the first blood circuit 6 of the patient P. The connecting device 80 disclosed herein is not required or provided in this case, nor is it part of the arrangement/set.

In the example of FIG. 1, the medical functional device 1 is further connected to, or comprises, a control device or closed-loop control device 150 for controlling or controlling in a closed-loop manner pumps, actuators, valves, throttles, tube clamps and/or similar and/or for performing at least the mechanical steps of the method according to the present disclosure for treating a patient P.

The extracorporeal blood circuit 6 of the patient P and the extracorporeal blood circuit 7 of the person D optionally each comprise an arterial tube clamp 62, 72. They each optionally further comprise an arterial connection needle (not shown in FIG. 1) connected to the arterial line 61, 71, or a connector (e.g., Luer lock) for connecting the arterial line 61, 71 to an arterial connection needle, respectively.

The blood circuit 6 and the blood circuit 7 each further comprise a venous tube clamp 64, 74. Optionally, they each comprise a venous connection needle (not shown in FIG. 1) connected to the venous line 63, 73, or a connector (e.g., Luer lock) for connecting the venous line 63, 73 to a venous connection needle, respectively.

In or on the blood circuit 6 of the patient P, a first optional blood pump 65 is arranged for conveying blood along the blood circuit 6, it is preferably arranged in or on its arterial line 61.

A second optional blood pump 75 is arranged in or on the blood circuit 7 of the person D for conveying blood along the blood circuit 7, it is preferably arranged in or on its arterial line 71.

Optionally, further pumps may be provided. In the embodiment of FIG. 1, a third blood pump 66 is exemplarily arranged in or on the venous line 63 of the blood circuit 6 of the patient P and/or a fourth blood pump 76 is also exemplarily arranged in or on the venous line 73 of the blood circuit 7 of the person D. Additionally or alternatively to these optional, further blood pumps 66, 76, there may be provided, respectively, devices for variably or adjustably modifying the flow or the flow area for shifting the blood volume, such as throttles.

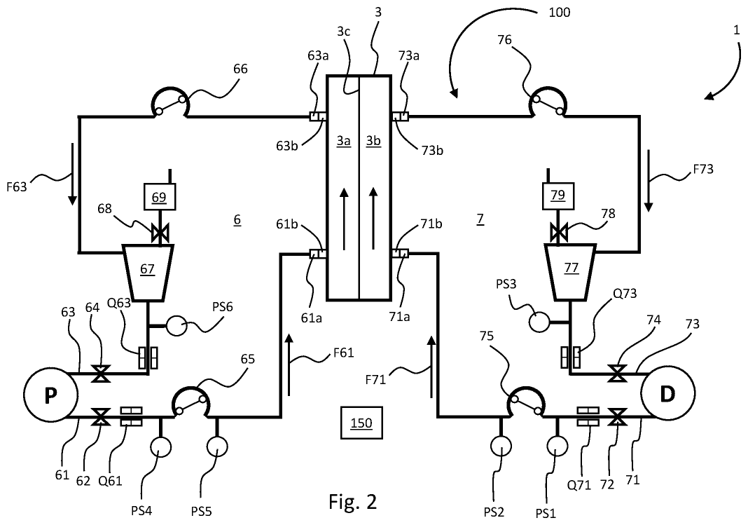

In the present example, the medical functional device 1 further comprises, here exemplarily four, flow sensors Q61, Q63, Q71 Q73, is in operative connection with them and/or is connected to them. In the example shown, one flow sensor is arranged on each of the arterial lines 61, 71 and on each of the venous lines 63, 73, respectively, in order to measure or determine the volume flows F61, F63, F71, F73 (see FIG. 2) that can be measured during use. At least one of the flow sensors Q61, Q63, Q71 Q73 may be designed as a magnetic-inductive flow sensor or as a section thereof. At least one of the flow sensors Q61, Q63, Q71 Q73 may be designed as at least a dual-channel flow sensor or a section thereof.

Optional balancing devices for balancing a conveyed volume along the respective blood circuit 6, 7 may be provided. They may comprise the control device or closed-loop control device 150, or be in signal communication therewith (not shown in FIG. 1). The flow sensors Q61, Q63, Q71 Q73, or any combinations thereof, may in some embodiments be part of the balancing device, or form it, together with the control device or closed-loop control device 150, or other computing device.

In some embodiments of the medical functional device 1, at least two of the flow sensors Q61, Q63, Q71, Q73 may be arranged in a common magnetic field. Alternatively or additionally, at least two sections of the first blood circuit 6 and/or two sections of the second blood circuit 7 are arranged such that to guide blood through one and the same magnetic field for at least two of the magnetic-inductive flow sensors Q61, Q63, Q71, Q73.

Further alternatively, a corresponding receptacle is provided for two sections of the first blood circuit 6 and/or the second blood circuit 7 in a common magnetic field existing during use of the medical functional device 1 or arrangement.

The control device or closed-loop control device 150 may be configured or programmed to be in signal communication, during use, with at least two of the flow sensors Q61, Q63, Q71, Q73 and to act in a closed-loop control manner on the first blood pump 65, the second blood pump 75, the optional further blood pumps 66 and/or 76 and/or the device (s) for variably modifying the flow in response to the signals transmitted by the flow sensors Q61, Q63, Q71, Q73.

The arrangement of FIG. 1 further comprises a plurality of optional pressure sensors PS1, PS5 (e.g., respectively upstream of the blood pumps 65, 75), PS2, PS4 (e.g., respectively downstream of the blood pumps 62, 75) which may exemplarily measure the pressure at the sites shown in FIG. 1 or at other sites. Additional or other pressure sensors may be provided, e.g., the pressure sensors PS3, PS6, positioned downstream of an optional venous bubble trap 67, 77, respectively. Corresponding de-aeration devices 69, 79 may be provided and may have further tube clamps 68, 78.

If the connectors 61a, 63a, 71a, 73a were now connected, e.g., in the manner described above (i.e., the connector 63a with the connector 73a or 71a, and the connector 61a with the connector 71a or 73a), and both the patient P and the person D were connected to the respective extracorporeal blood circuits 6, 7 by the connection needles, the extracorporeal blood circuit 6 of the patient P would be connected to the extracorporeal blood circuit 7 of the person D (see also FIG. 2). The blood circuits 6, 7 would thus be in both substance exchange and fluid exchange.

According to the present disclosure, it is provided that the connectors 61a, 63a, 71a, 73a may be directly connected to each other, or that one or two adapters are provided for this purpose, which may also be part of the arrangement or set. The function of the adapters may be limited to connecting lines and guiding liquid. In the case of direct connection, or connection via an adapter, however, not and thus differently than shown in FIG. 2 via a blood treatment component such as a dialyzer, the vascular system of the person D and the vascular system of the Patient P are completely (i.e., unfiltered), in substance exchange with each other.

In the extracorporeal blood circuit 6, blood from the patient P would then flow through the arterial line 61 to the venous line 73 of the extracorporeal blood circuit 7 of the person D and then into their vascular system to be purified by the natural bodily functions of the person D. Through the arterial line 71 of the extracorporeal blood circuit 7 of the person D and the venous line 63 of the patient P, purified blood would be returned to the patient P and introduced into their vascular system.

The medical functional device 1 may optionally be present, at least in sections thereof, in or on a blood cassette and/or be designed as a single-use item or a disposable, at least with regard to its first blood circuit 6, its second blood circuit 7 and the flow sensors Q61, Q63, Q71, Q73 as set forth herein.

It should be noted that the arterial lines 61, 71 shown in FIG. 1 are designated as arterial because it is assumed that they will, when in use, carry blood away from patient P and person D, respectively. However, their designation should not be understood as a limitation, at least as long as no patient P or person D is actually connected to them, which is evidently not yet the case in FIG. 1.

FIG. 2 shows in a simplified illustration a medical functional device 1 according to the present disclosure or an arrangement of components thereof in a further embodiment.

The arrangement in FIG. 2 corresponds in large parts to FIG. 1. For this reason, reference is made to the description with regard to FIG. 1 in order to avoid repetition. Preferably, the differences to FIG. 1 are discussed below.

The medical functional device 1 is shown in FIG. 2 in an at least partially equipped state of use. The patient P and the person D are connected to their respective extracorporeal blood circuits 6, 7 by double-needle access for blood treatment. All components to be flushed and/or primed prior to use, such as the aforementioned lines and the blood treatment component discussed below, have been flushed and/or primed as usual.

In other embodiments, using, e.g., additional Y-connectors (not shown in FIG. 2), the vascular systems of the patient P or of the person D may also be connected by single-needle access. Corresponding Y-connectors and/or other components required for this purpose may also be encompassed by the arrangement according to the present disclosure or by the set according to the present disclosure.

In the example of FIG. 2, the connecting device 80 comprises a blood treatment component 3, here exemplarily a blood filter (dialyzer). In some embodiments, the blood treatment component 3 may be part of the connecting device 80.

For its connection to the arterial line 61 and to the venous line 63 of the extracorporeal blood circuit 6 of the patient P, the blood treatment component 3 comprises corresponding connection sites 61b, 63b and a blood chamber 3a being in fluid communication therewith. Another chamber of the blood treatment component 3, used as blood chamber 3b, may be connected to the arterial line 71 and to the venous line 73 of the extracorporeal blood circuit 7 of the person D by suitable connection sites 71b, 73b.

Some or all of the connection sites 61b, 63b, 71b, 73b may be directly connectable to some or all of the connectors 61a, 63a, 71a, 73a. However, adapters or intermediate lines may also be provided for connecting connection sites to connectors.

The connectors 61a, 63a, 71a, 73a also do not necessarily have to be directly or indirectly connected to the connection sites 61b, 63b, 71b, 73b as shown in FIG. 2: if the connection as shown in FIG. 2 leads to a flow through the blood treatment component 3 in the parallel-current principle (via both connection sites 61b or 71b, arranged in FIG. 2 to be associated with the lower end of the blood treatment component 3, blood flows in, respectively, while it respectively flows out again via the connection sites 63b or 73b associated with the upper end of the blood treatment component 3), then the first blood circuit 6 and the second blood circuit 7 may, in several embodiments, alternatively be, or become, connected to each other such that blood flows through the blood treatment component 3 in a countercurrent principle (in that, for example, the connector 73a is connected to the connection site 71b rather than to the connection site 73b as shown in FIG. 2 and the connector 71a is connected to the connection site 73b rather than to the connection site 71b as shown in FIG. 2). In the case of a flow through the blood treatment component 3 in the countercurrent principle, the arrow pointing upward under the reference numeral 3b in FIG. 2 would then be pointing downward. In this case, it is at liberty not to interchange the connection sites 73a and 71b as described above in order to achieve a flow in the countercurrent principle, but rather alternatively to interchange the connection sites 61a and 63a in an analogous manner.

The blood chamber 3a and the blood chamber 3b are separated from each other by a mostly semi-permeable membrane 3c. In this embodiment, it represents the separating divide or parting between the extracorporeal blood circuit 6 of the patient P and the extracorporeal blood circuit 7 of the person D.

In this, the blood treatment component 3 may be a commercially available dialyzer, which according to the present disclosure, however, is not used for material exchange between blood and dialysis liquid, but for material exchange between the blood of the patient P and the person D. The provision, preparation, etc. of dialysis liquid may thus advantageously be omitted according to the present disclosure.

Optionally, in each of the arterial lines 61, 71 of the extracorporeal blood circuit 6, 7, the latter each have a flow sensor Q61, Q71 or are connected thereto. Optionally, in each of the venous lines 63, 73 of the extracorporeal blood circuit 6, 7, the latter have at least further flow sensors Q63, Q73 or are connected thereto. The flow sensors Q61, Q63, Q71, Q73 are optionally part of the functional device 1 according to the present disclosure.

The flow sensor Q61 is suitable and provided for measuring or detecting the first volume flow F61 (preferably in the dimension mass, or volume, respectively per time) in preferably the arterial line 61 of the extracorporeal blood circuit 6 of the patient P.

The flow sensor Q63 is suitable and provided for measuring or detecting the second volume flow F63 in preferably the venous line 63 of the extracorporeal blood circuit 6 of the patient P.

The flow sensor Q71 is suitable and provided for measuring or detecting the third volume flow F71 in preferably the arterial line 71 of the extracorporeal blood circuit 7 of the person D.

The flow sensor Q73 is suitable and provided for measuring or detecting the fourth volume flow F73 in preferably the venous line 73 of the extracorporeal blood circuit 7 of the person D.

The balancing device or the control device or closed-loop control device 150 may be programmed or provided in order to establish a fluid balance from some or all of these volume flows F61, F63, F71, F73 and, based on this established fluid balance, to control or to control in a closed-loop manner
at least one volume flow F61, F63, F71, F73 of the first blood circuit 6 of the patient P or of the second blood circuit 7 of the patient D, and/or
a pump rate of at least one of the blood pumps 65, 66, 75, 76 of the first blood circuit 6 of the patient P and/or of the second blood circuit 7 of the person D, and/or
at least one device for a variable or adjustable modification of the flow.

The blood flows of the patient P and of the person D are guided here optionally in the parallel-current principle through the blood treatment component 3, this is indicated in FIG. 2 by two co-running arrows. Toxins, water and similar may diffuse from the blood of the patient P into the blood of the person D and subsequently be processed or metabolized in the body of the person D.

Alternatively, guiding the blood flows through the blood treatment component 3 in the countercurrent principle is also encompassed by the present disclosure.

Since the blood of the patient P does not come into contact with the blood of the person D when a blood treatment component 3 is interposed, unlike the implementation of the present disclosure according to FIG. 1, the requirements for the person D in terms of compatibility of blood groups or similar may be considerably reduced.

Instead of a blood filter (dialyzer) as a blood treatment component, the interposition of a liver substitute apheresis adsorber or a blood oxygenator between the two blood circuits 6 and 7 or their connection to each other via this or therethrough is also encompassed by the present disclosure.

The arrows shown in FIG. 2 generally indicate each the direction of flow.

In addition or alternatively to the third and/or fourth blood pump 66, 76, the medical functional device 1 may comprise at least one device for variably or adjustably modifying the flow or flow area or for shifting the blood volume, which device is not a pump, for example a flow-pressure control component, in particular a throttle. Such a component is not shown in FIG. 2.

While FIG. 1 shows a functional device 1 or arrangement according to the present disclosure without it being in use, FIG. 2 already shows a blood treatment apparatus 100 based thereon.

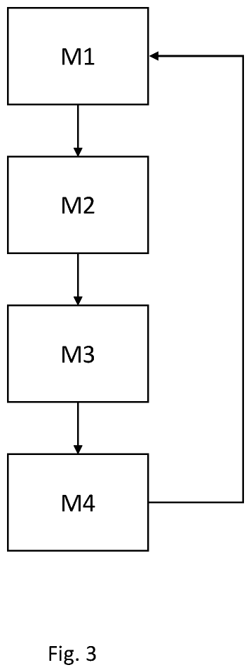

FIG. 3 shows in a simplified representation the course of a method according to the present disclosure for treating a patient P using the medical functional device 1, an arrangement of components thereof and/or a blood treatment apparatus 100 according to the present disclosure in an embodiment.

Method step M1 represents the optional determining of at least a first volume flow F61, F71 in at least the arterial line 61, 71 of the first blood circuit 6 or the second blood circuit 7 via the flow sensors Q61, Q71.

Method step M2 represents the optional determining of at least a second volume flow F63, F73 in at least the venous line 63, 73 of the first blood circuit 6 or the second blood circuit 7 via the flow sensors Q63, Q73.

The establishment of a fluid balance from at least the first volume flow F61, F71 and the second volume flow F62, F72 is represented by the optional method step M3.

The optional method step M4 represents controlling and/or controlling in a closed-loop manner
at least one volume flow F61, F63, F71, F73 of the first blood circuit 6 of the patient P or of the second blood circuit 7 of the person D, and/or
a pump rate of at least one of the blood pumps 65, 66, 75, 76 of the first blood circuit 6 of the patient P and/or of the second blood circuit 7 of the person D, and/or
at least one device for variably or adjustably modifying the flow
   based on the established fluid balance.

These steps can be repeated continuously during the treatment of a patient P in order to achieve the most accurate control/regulation of the medical functional device 1 and/or of its components, in particular of its blood pumps 65, 66, 75, 76 during the treatment.

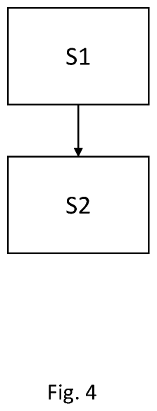

FIG. 4 shows in a simplified representation the course of a method according to the present disclosure for preparing an arrangement of components for treating a patient P by hemodialysis or exchange transfusion.

Reference is made to the reference numerals from the preceding figures.

In this, method step S1 represents the provision of an arrangement according to the present disclosure.

In method step S2, the first blood circuit 6 is connected to the second blood circuit 7 by the connecting device 80 and/or the first blood circuit 6 or the second blood circuit 7 is connected to the flow sensors Q61, Q63, Q71, Q73.

### LIST OF REFERENCE NUMERALS

1 medical functional device
3 blood treatment component; dialyzer
3a blood chamber
3b blood chamber
3c semi-permeable membrane
6 first blood circuit
7 second blood circuit
61 arterial line or arterial patient line
61a connector
61b connection site
62 arterial tube clamp
63 venous line or venous patient line
63a connector
63b connection site
64 venous tube clamp
65 first blood pump
66 third blood pump
67 venous bubble trap
68 tube clamp
69 de-aeration device
71 arterial line or arterial line of the renally not
impaired person
71a connector
71b connection site
72 arterial tube clamp
73 venous line or venous line of the renally not impaired person
73a connector
73b connection site
74 venous tube clamp
75 second blood pump
76 fourth blood pump
77 venous bubble trap
78 tube clamp
79 de-aeration device
80 connecting device
100 blood treatment apparatus
150 control device or closed-loop control device
D second renally not impaired person (short: person)
F61 first volume flow
F63 second volume flow
F71 first volume flow
F73 second volume flow
M1 method step
M2 method step
M3 method step
M4 method step
P patient
PS1 pressure sensor
PS2 pressure sensor
PS3 pressure sensor
PS4 pressure sensor
PS5 pressure sensor
PS6 pressure sensor
Q61 flow sensor
Q63 flow sensor
Q71 flow sensor
Q73 flow sensor
S1 method step
S2 method step

1. A medical functional device or arrangement, each comprising
a first blood circuit for withdrawing blood from a patient and reintroducing blood to the patient; and
a second blood circuit for withdrawing blood from a person that is not renally impaired and reintroducing blood to the person that is not renally impaired, wherein:
the first blood circuit and the second blood circuit each comprise an arterial line and a venous line; and
(i) the medical functional device or the arrangement comprises at least one connecting device for establishing fluid communication between the arterial line of the first blood circuit and the venous line of the second blood circuit and between the arterial line of the second blood circuit and the venous line of the first blood circuit, or (ii) the arterial line of the first blood circuit is connected to, or integral with, the venous line of the second blood circuit and the arterial line of the second blood circuit is connected to, or integral with, the venous line of the first blood circuit.

2. The medical functional device or arrangement of claim 1, wherein the connecting device comprises, or consists of, one or more connectors configured to establish the fluid communication between (i) the arterial line of the first blood circuit and the venous line of the second blood circuit and (ii) between the arterial line of the second blood circuit and the venous line of the first blood circuit.

3. The medical functional device or arrangement of claim 2, further comprising a blood treatment component connected to or configured to be connected to (i) the arterial line and the venous line of the first blood circuit and (ii) the arterial line and the venous line of the second blood circuit.

4. The medical functional device or arrangement of claim 3, wherein the blood treatment component is part of the connecting device and comprises one or more connection sites configured to be connected to the one or more connectors.

5. The medical functional device or arrangement of claim 1, further comprising a blood pump configured to convey blood along the arterial line of the first blood circuit.

6. The medical functional device or arrangement of claim 1, further comprising a blood pump configured to convey blood along the arterial line of the second blood circuit.

7. The medical functional device or arrangement of claim 5, further comprising a device configured to (i) variably modify a flow of blood along the venous line of the first blood circuit or shift a blood volume along the venous line of the first blood circuit or (ii) convey blood along the venous line of the second blood circuit.

8. The medical functional device or arrangement of claim 1, further comprising a first balancing device configured to balance a volume conveyed along the first blood circuit and/or a second balancing device configured to balance a volume conveyed along the second blood circuit.

9. The medical functional device or arrangement of claim 8, wherein the first balancing device and/or the second balancing device comprises at least one flow sensor, wherein the at least one flow sensor is embodied and/or arranged for measuring a flow of the volume conveyed along the first blood circuit and/or a flow of the volume along the second blood circuit.

10. The medical functional device or arrangement of claim 9, wherein the at least one flow sensor comprises a magnetic-inductive flow sensor or a section thereof.

11. The medical functional device or of claim 9, wherein the at least one flow sensor comprises an at least two-channel flow sensor.

12. The medical functional device or arrangement of claim 10, wherein (i) the at least one flow sensor comprises at least two flow sensors arranged in a common magnetic field, or (ii) at least two sections of the first blood circuit and/or at least two sections of the second blood circuit are arranged to guide blood through a common magnetic field for at least two magnetic-inductive flow sensors of the at least one flow sensor or a corresponding reception of the two sections of the first blood circuit and/or the two sections of the second blood circuit is provided in a common magnetic field existing during use of the medical functional device or arrangement.

13. The medical functional device or arrangement of claim 9, wherein the first balancing device and/or the second balancing device is connected to a control device configured or programmed to be in signal communication with at least two flow sensors of the at least one flow sensor and to act, as a response to signals transmitted by at least two of the flow sensors, in a closed-loop manner on a first blood pump configured to convey blood along the arterial line of the first blood circuit, on a second blood pump configured to convey blood along the arterial line of the second blood circuit, and/or on a device for variably modifying a flow of blood along the venous line of the first blood circuit.

14. The medical functional device or arrangement of claim 1, wherein the medical functional device is embodied at least in sections as a cassette or is present thereon or therein.

15. A balancing device arranged, or configured to be arranged, on both an arterial line and a venous line of a blood circuit, the balancing device configured for determining at least one fluid balance between the arterial line and the venous line,
wherein the balancing device comprises or is connected to at least two flow sensors arranged for determining a blood flow in the arterial line or in the venous line of the blood circuit.

16. The balancing device of claim 15, wherein the balancing device further comprises or is connected to a control device, the control device being configured or programmed to act, in a closed-loop manner in response to signals transmitted by the at least two flow sensors, on a blood pump to regulate the flow through the blood circuit.

17. A blood treatment apparatus for treating a medical fluid with at least one balancing device according to claim 16.

18. A blood treatment apparatus for treating a medical fluid with at least one medical functional device according to claim 1.

19. A method for preparing an arrangement of components for treating a patient by hemodialysis or exchange transfusion, the method comprising:
providing an arrangement according to claim 1; and
connecting the first blood circuit via the connecting device to the second blood circuit and/or connecting the first blood circuit or the second blood circuit to at least one flow sensors.

20. A method for treating a patient using the medical functional device or arrangement of claim 1 and/or a blood treatment apparatus of claim 18.

21. The method of claim 20, further comprising:
determining, using at least two flow sensors, a first volume flow in at least the arterial line of the first blood circuit or in the arterial line of the second blood circuit;
determining, using the flow sensors, a second volume flow in at least the venous line of the first blood circuit or the venous line of the second blood circuit;
establishing a fluid balance from at least the first volume flow and the second volume flow; and
based on the established fluid balance, controlling at least one of: a volume flow of the first blood circuit or of the second blood circuit, a pump rate of at least one blood pump of the first blood circuit and/or of the second blood circuit, or at least one device for variably modifying the flow.

22. A method for differential balancing blood of a patient, the method comprising differentially balancing the blood of the patient using (i) a magneto-inductive fluid balancing device comprising a magneto-inductive flow sensor and two flow channels or (ii) a laser-based fluid balancing device.
